(19) United States
(12) Patent Application Publication
(10) Pub. No.: US 2011/0125148 A1
(43) Pub. Date: May 26, 2011

(54) Multiple Frequency Energy Supply and Coagulation System

(76) Inventors: Paul F. Turner, Bountiful, UT (US); Mark Hagmann, West Valley, UT (US)

(21) Appl. No.: 13/020,483

(22) Filed: Feb. 3, 2011

Related U.S. Application Data
(63) Continuation-in-part of application No. 12/620,002, filed on Nov. 17, 2009.
Application No. 12/620,002 is a continuation-in-part of application No. 12/689,195, filed on Jan. 18, 2010.
Application No. 12/689,195 is a continuation-in-part of application No. 12/794,667, filed on Jun. 4, 2010.

(51) Int. Cl. A61B 18/18

(52) U.S. Cl. 606/33

(57) ABSTRACT

An electromagnetic energy supply system for supplying electromagnetic energy signals to an applicator for applying the electromagnetic energy signals to tissue for heating such tissue can supply electromagnetic energy signals of at least one selected frequency of a plurality of frequencies available for selection. The system includes a plurality of individual electromagnetic signal generators each adapted to generate signals of a particular frequency, such as two microwave energy signal generators with one operating at 915 MHz and one operating at 1450 MHz. A system controller coordinates operation of the generators. Each generator is connected to connectors for connecting an applicator or array of applicators for applying the selected frequency electromagnetic energy signals to the tissue to be heated. A power splitting circuit can provide multiple connectors tuned for one or for more than one applicator to be connected. The system can provide a treatment using a single selected frequency, a plurality of selected frequencies simultaneously, or can switch between selected frequencies.

### RELATED APPLICATIONS

This is a continuation-in-part of co-pending application Ser. No. 12/620,002, filed Nov. 17, 2009, of co-pending application Ser. No. 12/689,195, filed Jan. 18, 2010, both entitled Microwave Coagulation Applicator and System, of co-pending application Ser. No. 12/794,667, filed Jun. 4, 2010, entitled Microwave Coagulation Applicator and System with Fluid Injection, and of PCT Application No. PCT/US2010/57127, filed Nov. 17, 2010, entitled Microwave Coagulation Applicator and System, all hereby incorporated herein by reference.

### BACKGROUND OF THE INVENTION

1. Field

This invention relates to heat treatment of living body tissue such as tissue ablation, coagulation, and hyperthermia, and particularly to the creation of heat in such tissue by the application of electromagnetic (EM) energy, such as microwave energy and/or radiofrequency energy, to such tissue. Further, the invention relates particularly to energy supply systems for generating desired electromagnetic energy signals and supplying such generated electromagnetic energy signals to one or more applicators which then supply the generated electromagnetic energy signals to the body tissue to be treated.

2. State of the Art

The use of electromagnetic (EM) energy, such as radiofrequency and microwave energy, to heat tissue for the treatment of disease is known. In using electromagnetic energy for tissue heating, an applicator for applying electromagnetic energy signals is positioned, or an array of such applicators are positioned, with respect to the tissue to be treated (heated) so that electromagnetic energy signals from the applicator or applicators penetrate and heat the tissue to be treated. Electromagnetic energy signals are generated and supplied to the one or more applicators by an electromagnetic energy signal supply system to which the one or more applicators are connected, generally by a connecting cable, such as a flexible coaxial cable. The electromagnetic energy signal supply system controls various parameters, such as the frequency and power level, of the electromagnetic energy signals supplied to the one or more applicators. Many electromagnetic energy signal applicators and electromagnetic energy signal supply systems are known in the art.

Death, or necrosis, of living tissue cells occurs at temperatures elevated above a normal cell temperature for a sufficient period of time. The sufficient period of time is generally dependent upon the temperature to which the cells are heated. Above a threshold temperature of about 41.5 degrees C., substantial thermal damage occurs in most malignant cells if this temperature is maintained for a sufficiently long time, typically longer than about 120 minutes. At temperatures above about 45 degrees C. thermal damage occurs to most normal cells if exposure exceeds about 60 minutes. During treatment, it is desirable to produce an elevated temperature within the targeted tissue for a time period sufficient to cause the desired cell damage, while keeping nearby healthy tissue at a safe lower temperature. For this reason, when treatment involving tissue heating is used, it is important to assure both adequate heating throughout the diseased tissue to be treated, such as a tumor, to the margin of the diseased tissue, such as to the tumor margin, and reduced heating and temperatures in the critical normal tissue surrounding the diseased tissue, so as to not damage such normal tissue.

Heating therapy is sometimes combined with other treatments, such as surgery, ionizing radiation, and chemotherapy. For example, when heating is combined with radiation, it is desirable to maintain the temperature within the diseased tissue within the range of about 42 to about 45 degrees C. Higher temperatures are usually undesirable when a combined treatment modality is used because higher temperatures can lead to microvessal collapse causing resistance to radiation therapy and decreasing the amount of systemic chemotherapy reaching the tumor if the tumor has suffered vascular damage. Lower temperatures are undesirable because they can fail to provide adequate therapeutic effect. Therefore, it is important to control the temperature in the treated tissue within the desired range for multi-modality treatments and not allow heating of the tissue in the tumor or around the tumor to above 45 degrees C. where tissue damage from other treatments may be compromised. Treatment within this controlled temperature range is now usually referred to as hyperthermia.

Forms of thermal therapy that kill the tissue with heating alone are generally referred to as coagulation or ablation. To adequately eradicate a cancerous tumor with only the application of heat, it is necessary to ensure adequate heating is accomplished throughout the entire tumor. In cases of a malignant tumor, if viable tumor cells are left behind, the tumor can rapidly grow back leaving the patient with the original problem. In what is generally referred to as coagulation or ablation, the diseased tissue is heated to at least about 55 degrees C., and typically above about 60 degrees C., for exposure times sufficient to kill the cells, typically for greater than about one minute. With coagulation and ablation treatments, there is a volume reduction of temperature that ranges from the high temperature in the treated tissue to the normal tissue temperature of 37 degrees C. outside the treated tissue. The outer margin of the overall heat distribution in the treated tissue volume may then result in damage to normal tissue if such normal tissue is overheated. Therefore, for prolonged coagulation or ablation treatments where the coagulation or ablation volume is maintained at very high temperatures, there is a high risk of damage to surrounding normal tissues.

For proper treatment of targeted cancerous tumor volumes or other tissue volumes to be treated using coagulation or ablation treatments, it becomes very important to properly deliver the correct thermal distribution over a sufficient time period to eradicate the tumor tissue while minimizing damage to critical surrounding normal tissue. With some tumor locations, such as in the liver, limited areas of normal tissue in which the tumor resides can be destroyed along with the tumor tissue by the heating without affecting the health of the patient. In such situations the coagulation can be applied in an aggressive way to include a margin of safety in destruction of limited surrounding normal tissues to assure that all of the cancerous tumor is destroyed. However, even in these locations, it is important to limit and control the destruction of normal tissue surrounding the tumor or other tissue to be treated.

The process of heating very rapidly to high temperatures that is common in coagulation and ablation treatments may utilize a rather short exposure time. In doing so, the resulting temperature distribution becomes primarily a result of the power absorption distribution within the tissue. However, if such treatments continue for multiple minutes, the blood flow and thermal conduction of the tumor and surrounding tissues will modify the temperature distribution to result in a less predictable heat distribution because the changes occurring in blood flow in such a heated region may not be predictable. Therefore, it is important to optimize the uniformity of the tissue heating power that is absorbed to lead to a more predictable temperature distribution that better corresponds with the treatment prescription. Therefore, pretreatment planning practices prior to treatment for calculating a predicted power and temperature distribution resulting from the parameters of power and relative phase of the energy applied to the tissue can be important for not only coagulation and ablation, but also hyperthermia. As higher temperatures are used during treatment, such higher temperatures may increase patient discomfort and pain, so it can be helpful to avoid excessive temperatures to reduce the need of patient sedation. Typically, diseased tissue such as cancerous tumors may not have a significant pain sensation, but normal tissues would be more likely to have pain limitations on treatment, particularly skin tissue of the patient.

Invasive electromagnetic energy applicators can be inserted into living body tissue to place the source of heating into or adjacent to a diseased tissue area. Invasive applicators help to overcome some difficulties that surface applicators experience when the target tissue region is located below the skin (e.g., the lung, prostate, or liver). Invasive applicators must be properly placed to localize the heating to the vicinity of the desired treatment area. Even when properly placed, however, it has been difficult to ensure that adequate heat is developed in the diseased tissue without overheating surrounding healthy tissue. Further, with applicators operating at higher power levels to produce the needed higher temperatures for coagulation and ablation, there is a tendency for the cable in the portion of the applicator leading from outside the body to the location of the application of the electromagnetic energy in the applicator to heat to undesirably high temperatures which can cause thermal damage to the normal tissue through which the applicator passes to reach the diseased tissue to be treated. Therefore, various ways of cooling the applicator have been used in the prior art.

A consideration in providing desired heating to diseased tissue while limiting the heating of the normal tissue surrounding the diseased tissue being treated is the shape and size of the heating pattern produced by the one or more antennas providing the electromagnetic energy signals to the tissue compared to the shape of the mass of diseased tissue to be treated. It has been found that different frequencies of electromagnetic energy signals produce different shapes and sizes of heating patterns. When referring to electromagnetic energy signals in the medical field, particularly such signals when applied to tissue to create heating of the tissue for ablation and coagulation treatments, frequencies of between about 200 KHz to about 13 MHz are usually referred to as radiofrequency (RF) signals. Frequencies above about 300 MHz are usually referred to as microwave signals. However, many references will refer to any electromagnetic energy signals used for producing heat in tissue as radiofrequency signals. When using the lower frequency signals, now generally referred to as radiofrequency signals, the radiofrequency electromagnetic signals are generally applied to the tissue by an applicator first electrode from which the signals enter the tissue and then flow to a second electrode. The second electrode can be located on the applicator, can be located as a separate invasive electrode inserted into the body tissue, or can be located on the outside of the body in contact with the skin. When using the higher frequency signals, usually referred to as microwave frequency signals, the applicator includes an antenna which radiates the microwave electromagnetic energy into the tissue to be treated. Various radiofrequency and microwave frequency applicators are currently known and commercially available.

Within the microwave frequency range, microwave frequencies of 915 MHz and 2450 MHz are approved for medical hyperthermia and ablation use. Microwaves of 915 MHz frequency are longer than microwaves of 2450 MHz frequency. The operating frequency of 915 MHz corresponds with a wavelength of 4 to 4.7 cm (depending on the published reference source and the dryness of the tissue) in typical high water content body tissues such as liver, muscle, and organs, whereas the operating frequency of 2450 MHz corresponds with a wavelength of 1.6 to 1.8 cm in typical high water content body tissues such as liver, muscle, and organs. Generally, it has been found that the longer 915 MHz frequency microwaves will penetrate more deeply into tissue and have less attenuation as they penetrate into the tissue, thereby producing a larger heating pattern at a given power level, than do microwaves of the higher 2450 MHz frequency. For the 915 MHz frequency waves, the plane-wave penetration to about a 13% power level is 4.2 to 4.3 cm while the 2450 MHz frequency plane-wave penetration to about a 13% power level is 2.1 to 2.2 cm. However, while the longer 915 MHz microwaves produce a larger heating pattern, the heating pattern produced by the currently available 915 MHz frequency microwave applicators are elliptical in shape and tend to be elongated along the applicator back from the antenna toward the outside of the body. The radial diameter of the heating or ablation zone for a single 915 MHz microwave antenna can be as little as half the length of the heating or ablation zone along the applicator. Currently available 2450 MHz frequency microwave applicators also produce a somewhat elliptical heating pattern, however the heating pattern produced by the 2450 MHz frequency microwave applicators tend to be less elliptical and more spherical than the heating pattern produced by the 915 MHz frequency applicators. Thus, the 915 MHz provides deeper penetration and longer coagulation length and 2450 MHz provides more shallow penetration and a shorter coagulation length. See, for example, Sun, et al., Comparison of Ablation Zone Between 915- and 2,450-MHz Cooled-Shaft Microwave Antenna: Results in In Vivo Porcine Livers, DOI:10.2214/AJR.07.3828 and AJR 2009; 192:511-514; and Liu et al., Comparison of percutaneous 915 MHz microwave ablation and 2450 MHz microwave ablation in large hepatocellular carcinoma, Int. J. Hyperthermia, 2010, 1-8, Early Online.

In testing and simulations performed by applicant, applicant has found that for single antenna applicator applications, the use of 2450 MHz provides a shorter ablation and heating length along the single inserted shaft than when using a single antenna applicator at 915 MHz. This results in an ablation zone that is more spherical for 2450 MHz than for 915 MHz for single applicator ablations. Since cancerous tumors to be treated are often substantially spherical in shape, the use of 2450 MHz appears to be advantageous when a small and more spherical ablation pattern is desired, particularly when using a single applicator. However, applicant has also found that when multiple applicators are used in a phased array arrangement, a larger and more spherical ablation and heating pattern can be produced than with arrays of multiple antennas operated at 2450 MHz, or than with single applicator ablation at either 915 MHz or 2450 MHz.

In addition, the lower frequency signals referred to above as the radiofrequency signals, typically lower than about 40 MHz, depending upon the configurations of the applicators and the specific frequencies used with the applicators, will also offer a variety of heating patterns. It has been found that these heating patterns will usually be smaller and more spherical than the heating patterns produced by one 915 MHz microwave applicator and smaller than an array of a plurality of 915 MHz microwave applicators.

The above suggests that, depending upon the size and shape of the mass of diseased tissue to be treated, there may be an advantage to the use of 2450 MHz microwaves in some situations, to the use of radiofrequency signals in other situations, and to the use of 915 MHz microwaves in still other situations. Therefore, in some situation it may be desirable to treat a patient using a single microwave applicator with microwaves of 2450 MHz while in other situations it may be more desirable to treat a patient using a single microwave applicator, or an array of microwave applicators, at 915 MHz. Further, in still other situations, it may be more desirable to treat a patient using a particular radiofrequency applicator using signals of a particular radiofrequency. Both 2450 MHz and 915 MHz microwave tissue treatment systems, as well as a number of radiofrequency tissue treatment systems, are currently commercially available. Therefore, once a determination is made as to the most desirable frequency to use for a particular treatment, a system providing the desired frequency has to be found. Electromagnetic energy tissue treatment systems are generally expensive and are generally large taking up limited space in a treatment area. Typically, tissue ablation and coagulation procedures are performed in a crowded room containing an imaging system, such as a computed tomography (CT) imaging system, an ultrasound (US) imaging system, a magnetic resonance imaging (MRI) system, or a sonographic imaging system, which is used to guide the placement of the applicator or applicators into the tissue to be treated, as well as the electromagnetic ablation system used for the treatment. Where an option of using either a 2450 MHz or a 915 MHz system is to be provided, a treatment center has to have two complete separate microwave ablation systems available for use. Further, if the option of using other frequency systems, such as other microwave frequencies or radiofrequencies, is to be provided, again separate complete systems have to be available for use. However, many treatment centers will not be able to afford having two or more separate complete systems or may not have the space for two or more separate complete systems. Further, when separate systems are used, such systems will generally have different operating control systems which require operator knowledge of each separate system or different operators may be required for each system. In addition, in many instances, it is not until the time of treatment that the patient is imaged using either ultrasound or CT to determine the size and location of the target tissue or tumor. This imaging then leads to the decision of what frequency and equipment would be best to use. It is very inconvenient in such a critical procedure not to have the desired ablation equipment in-place and ready for treatment use prior to the pretreatment images taken just prior to treatment. However, having two or more different and separate systems ready for use is not generally practical.

### SUMMARY OF THE INVENTION

According to the invention, an electromagnetic energy heat treatment system for heating body tissue in need of treatment includes a single electromagnetic energy signal supply system which can provide a choice of electromagnetic energy signals of two or more frequencies to one or more applicators adapted to apply electromagnetic energy signals of a selected frequency to body tissue in need of treatment. An embodiment of an electromagnetic energy supply system that can provide a choice of two or more frequencies of electromagnetic energy signals includes a first electromagnetic energy signal generator adapted to provide electromagnetic energy signals of a first frequency, a second electromagnetic energy signal generator adapted to provide electromagnetic energy signals of a second frequency, and, if desired, additional electromagnetic energy signal generators to provide electromagnetic energy signals of additional frequencies, all generators having a common power supply and a common system controller. The system also includes electromagnetic energy signal output connectors for connecting applicators adapted for applying electromagnetic energy signals of a particular frequency supplied by the system to tissue to be treated. For example, where the system includes first and second electromagnetic energy signal generators, the system would include signal output connectors for an applicator adapted for applying electromagnetic energy signals of the first frequency to tissue to be treated and/or for one or more applicators adapted for applying electromagnetic energy signals of the second frequency to tissue to be treated. The system may be such as to provide a selected one or the other frequency for a particular treatment procedure, or may be adapted to provide an output of both frequencies simultaneously or multiplexed to provide both frequencies on a substantially simultaneous basis, or on a timed basis where one frequency is provided for a predetermined time interval and the other frequency is then provided for a predetermined time interval.

While the two or more electromagnetic energy signal generators of the system can provide electromagnetic energy signals of two or more of any microwave or radiofrequency frequency, the invention will be illustrated and described in detail in connection with an example embodiment which provides electromagnetic energy signals in the form of microwave energy signals at a selected 2450 MHz and/or 915 MHz. An embodiment of a microwave energy supply system that can provide a choice of electromagnetic energy signals of 2450 MHz and/or 915 MHz includes both a 915 MHz microwave generator and a 2450 MHz microwave generator with a common power supply and common controller to provide output connections for an applicator adapted for applying microwaves of 2450 MHz frequency to tissue to be treated and/or for one or more applicators adapted for applying microwaves of 915 MHz frequency to tissue to be treated. The system may be such as to provide a selected one or the other frequency for a particular treatment procedure, or may be adapted to provide an output of both frequencies simultaneously or multiplexed to provide both frequencies on a substantially simultaneous basis, or on a timed basis where one frequency is provided for a predetermined time interval and the other frequency is then provided for a predetermined time interval.

A system of the invention, as described above, can provide the use of either 915 MHz microwaves or 2450 MHz microwaves during different treatment procedures or can provide both 915 MHz microwaves and 2450 MHz microwaves during the same treatment procedure under common control by the single controller. Typically, when treating cancerous tumors, there may be multiple tumors where some are large and some are small. A combined frequency system enables patient set up for multiple tumors or even extended and larger portions of tumors to be treated using both frequencies simultaneously, multiplexed or time switched between frequencies, or sequentially with the same patient treatment set-up and system. Further, the system can provide independent automatic control and treatment of different target tumor zones automatically controlled by the common system controller, typically a digital computer, using a common interface to provide an operator with much easier and more practical treatment planning, set-up, and delivery. Having a fully integrated system with the same power supply, computer controller, computer interface display, computer control software, and fans for providing cooling air flow to the single power supply all lead to major cost savings and space savings compared to having a separate system for each frequency. Further, the system can provide the advantages of using common tissue temperature monitoring and control, maintaining treatment data records, and reduced sedation times for the patient with more rapid treatment delivery. Any applicator adapted for use with 2450 MHz can be connected to the 2450 MHz power output connector and any applicator or applicators adapted for use with 915 MHz can be connected to the 915 MHz power output connectors. A 915 MHz applicator satisfactory for use with the system is shown and described in detail in copending parent application Ser. No. 12/620,002 incorporated herein by reference.

In one embodiment of the present invention, the microwave energy supply system includes a 915 MHz signal generator, a 2450 MHz signal generator, a power supply for supplying power to the signal generators, a computer controller and controller interface for controlling system operation, and a 915 MHz power splitter allowing the connection of one to three 915 MHz applicators to the system. In this embodiment, a single microwave power output connector is provided for connection of a single 2450 MHz applicator, when desired to apply 2450 MHz microwave energy to tissue to be treated, and four microwave power output connectors are provided through the power splitter for connection of up to three 915 MHz applicators when desired to apply 915 MHz microwave power either through a single applicator or simultaneously through an array of two or three applicators. With this arrangement one of the four 915 MHz output connectors is provided as a single connection output connector for use when a single 915 MHz microwave applicator is used, and three of the four 915 MHz output connectors are provided as multiple connection output connectors for connection of 915 MHz applicators to either two or three of the multiple output connectors. The power splitter is an impedance matched splitter circuit which automatically allows the power to be directed to the connection of 1, 2, or 3 applicators while maintaining an impedance match between the 915 MHz signal generator and the one, two, or three applicators for efficient power transfer from the generator to the applicator or applicators. If more than three applicators are desired for an array, additional power splitters can be included in the system and connected in parallel to the 915 MHz signal generator. For example, if two power splitter circuits are used anywhere between one and six applicators can be connected to the system.

In the one embodiment, the 915 MHz signal generator and the 2450 MHz signal generator can each be separate narrow band tuned microwave generators that can provide relatively high efficiency of input vs. output power of typically 50% to 70%. A generator that is variable in frequency, such as a tunable broadband generator which can be adjusted over a frequency range that would include 915 MHz and 2450 MHz typically operates at much lower efficiency between about 6% to possibly 20%. Thus, the use of the separate narrow band tuned microwave generators provides much higher power efficiency to the system. The poor efficiency of the variable frequency microwave power generator requires much higher input power which means that the power supply supplying the input power to such a variable frequency signal generator would need to be more than 3 times larger and higher rated than a power supply supplying a single narrow band generator, and still much larger than a power supply capable of supplying power to two narrow band generators simultaneously. The larger the power supply, the more heat generated which requires more airflow cooling and fan noise to remove the extra internally generated heat, and the more heat being discharged into and heating the treatment room. The increased system efficiency provided by the increased efficiency of the narrow band tuned microwave generators allows much smaller equipment packaging, lower cooling noise, and less heat discharged into the treatment environment.

While the system can provide for simultaneous operation of both the 915 MHz signal generator and the 2450 MHz signal generator so that, if and when desired, both 915 MHz and 2450 MHz microwave power can be supplied to both the 915 MHz applicator or applicators and the 2450 MHz microwave applicator simultaneously, such a system requires a power supply having sufficient power output to power both generators at their maximum power requirement simultaneously. Substantially the same results can be obtained by operating only one of the microwave signal generators at a time, with the capability of quickly switching between the signal generators, thereby lessening the power output requirements for the power supply and the overall system. Since the tissue heating is a result of the average power absorption in the tissue, it does not matter whether the power applied is pulsed, time sequenced, or continuous wave if the average power is the same and the time cycle is not more than up to about twenty seconds. With such an arrangement, a physician can install both 915 MHz and 2450 MHz applicators during an applicator positioning period and have the system ready to apply treatment through an array of 915 MHz antennas and sequentially or periodically through the 2450 MHz antenna. This can be provided by a timed switching between the two signal generators, or switching under manual control when the physician is ready to switch to the other antenna and frequency. This can be done quickly moving from one treatment field to the other possibly neighboring field where a rapid back and forth switching (multiplexing) allows the ablated zone of the first treatment to retain its temperature while applying microwave energy to the other, and back and forth to maintain the treatment temperature in both fields or zones. If time switched during the same procedure, the whole region will remain at ablation temperatures provided by time gated switching between the two frequency modes to provide a larger and more tailored ablation pattern than that which the phased array at 915 MHz might achieve alone. This can also provide for simultaneous heating of smaller tumors at 2450 MHz while larger tumors are treated at 915 MHz by having the system doing time switching of its operation between the two frequency operating modes. This switching is possible only with the provision of the two separate generators to which the appropriate applicators can be connected through separate connections and both generators are controlled by a common controller.

Most ablation or coagulation systems include temperature sensors and/or other sensors to provide feedback for control of the electromagnetic energy signal supply system. For example, with the 915 MHz applicator as shown in copending parent application Ser. No. 12/620,002, a temperature sensor is provided in the cooling fluid channel of the applicator to measure the temperature of the cooling fluid which provides an indication of whether or not the applicator cooling system is operating properly. Further, in many systems, temperature sensors can be positioned in the tissue to be heated to indicated the tissue temperature with the temperature measured used as a control for the amount of power applied to the tissue to control the temperature of the heated tissue and/or in the tissue at the outer margin of the tissue to be heated to indicate when the temperature of the normal tissue to be protected is close to being overheated so protective measures need to be taken. In any event, the system of the invention can include any of these feedback, monitoring, and safety features. Where temperature or other sensors are inserted into a patient, an advantage of the system of the invention is that once such sensors are in the patient's body, they can remain to monitor various tissue variables, such as tissue temperature, including temperature in sensitive normal tissues to prevent over temperature exposure, whether using the first or the second frequency applicators in the same procedure or in different procedures performed substantially sequentially.

### THE DRAWINGS

Other features of the invention will become more readily apparent from the following detailed description when read in conjunction with the drawings in which the accompanying drawings show the best modes currently contemplated for carrying out the invention, and wherein:

FIG. 1 is a block diagram showing an embodiment of the dual frequency ablation system of the invention;

FIG. 2 is a side elevation of an applicator, in accordance with an embodiment of the present invention;

FIG. 3 is a vertical section of a portion of the applicator of FIG. 2;

FIG. 4 is a vertical sectional of the handle portion of the applicator of FIG. 2;

FIG. 5 is a circuit diagram of the electrical connections within the handle portion of the applicator as shown in FIG. 4; and

FIG. 6 is a block diagram of a power splitter of the invention.

### DETAILED DESCRIPTION OF THE ILLUSTRATED EMBODIMENTS

Reference will now be made to the exemplary microwave energy signal embodiments illustrated in the drawings, and specific language will be used herein to describe the same. It will nevertheless be understood that no limitation of the scope of the invention is thereby intended. For example, any combination of microwave and/or radiofrequency energy signals can be used. Alterations and further modifications of the inventive features illustrated herein, and additional applications of the principles of the inventions as illustrated herein, which would occur to one skilled in the relevant art and having possession of this disclosure, are to be considered within the scope of the invention.

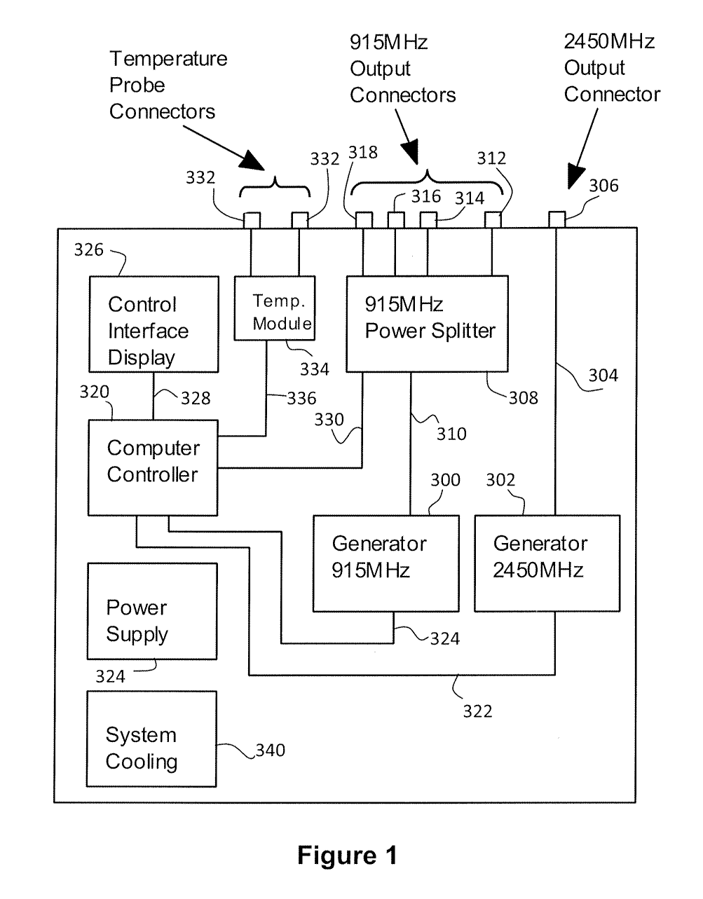

FIG. 1 is a block diagram showing an embodiment of the microwave energy supply system of the invention. Referring to FIG. 1, an example embodiment of the microwave energy supply system of the invention includes a 915 MHz signal generator 300 and a 2450 MHz signal generator 302. Each of these generators can be operated to generate microwave signals of the indicated frequency in a form to be transmitted to a microwave applicator adapted to be inserted into a body in position to provide the microwave signals to tissue in the body to be heated to a temperature sufficient to provide a desired treatment to the tissue. For example, the tissue to be treated may be a cancerous tumor and the heating provided by the microwave energy heats the tissue to a temperature sufficient to ablate (kill or coagulate) the diseased tissue.

As indicated above, it has been found that with currently available microwave applicators, when a single 2450 MHz applicator is used for tissue treatment, a small but close to spherical heating pattern is created in the tissue around the applicator. A small but close to spherical heating pattern can also be created with lower frequency radiofrequency signals. When a single 915 MHz applicator is used, the heating pattern is typically larger, but is ellipsoidal in shape. Further, applicant has found that when multiple applicators are used in a phased array arrangement and simultaneously operated at 915 MHz, a larger and more spherical ablation and heating pattern can be produced than with a single applicator operated at either 915 MHz or 2450 MHz, or than can be produced with an array of multiple applicators operated at 2450 MHz. Since cancerous tumors to be treated are often substantially spherical in shape, the use of a single 2450 MHz applicator (or a lower frequency radiofrequency applicator) appears to be advantageous when a small and more spherical ablation pattern is desired. However, when a larger and more spherical ablation pattern is desired, it is advantageous to use multiple 915 MHz applicators in a phased array arrangement. Therefore, to be able to provide a wide range of ablation patterns, it would be advantageous in a microwave treatment system to provide a microwave treatment system that can provide single applicator 2450 MHz ablation and/or either single applicator 915 MHz ablation or multiple applicator 915 MHz phased array ablation.

Referring again to FIG. 1, the output of the 2450 MHz signal generator 302 is connected by cable 304, such as a coaxial cable, to a single 2450 MHz microwave output connector 306, such as a coaxial cable connector, to which a flexible coaxial cable, not shown, can be connected to connect the output of 2450 MHz signal generator 302 to a single 2450 MHz applicator, not shown.

The output of the 915 MHz signal generator 300 could also be connected directly to a single 915 MHz microwave output connector to which a flexible coaxial cable could be connected to connect the output of 915 MHz signal generator 300 directly to the single 915 MHz applicator. However, this would only allow use of a single 915 MHz applicator. Since, as indicated, it has been found advantageous in many treatment situations to provide an array of a plurality of 915 MHz applicators, it has been found advantageous to connect the output of 915 MHz signal generator 300 to a plurality of 915 MHz output connectors so a plurality of 915 MHz applicators can be connected, if desired, and operated as an array.

In the illustrated embodiment, which provides for connection of a single 915 MHz microwave applicator or for connection of a plurality of 915 MHz microwave applicators to be operated in a phased array, the single 915 MHz microwave generator 300 is connected to a passive, non-switching, microwave impedance matched power splitter (divider) 308 which is used to direct power simultaneously to multiple connectors 312, 314, 316, and 318 to which one or more 915 MHz microwave applicators can be connected. This arrangement provides approximately equal power simultaneously to each of the output connectors. This arrangement also provides equal phase output of the microwave energy at each of the output connectors. Thus, when multiple antennas are connected to the connectors of the power splitter, they have equal power and equal relative phase and are thus correctly called a phased array of antennas. The cables going to the radiating points on each antenna are maintained at the same electrical length so that the radiated energy from the antennas are phase synchronous and phase coherent. Phase synchronous meaning that there is a fixed phase relationship between the radiation phase of all antennas and phase coherent meaning that the relative radiated phase from each antenna is approximately the same. Since different array patterns are desirable for different optimized treatments, and desired treatments can use a single 915 MHz applicator or varying numbers of multiple 915 MHz applicators, the 915 MHz power splitter is adapted to power and monitor a single applicator or a multiple number of applicators.

As shown in FIG. 1, the output of 915 MHz signal generator 300 is connected to 915 MHz power splitter 308, through cable 310, such as a coaxial cable. In the illustrated embodiment, 915 MHz power splitter 308 provides four 915 MHz microwave power output connectors 312, 314, 316, and 318. Up to three 915 MHz applicators can be connected via flexible coaxial cables, not shown, to selected 915 MHz microwave power output connectors 312, 314, 316, and 318, thereby connecting the up to three 915 MHz applicators through the cables and power output connectors to the 915 MHz power splitter, when desired to apply 915 MHz microwave power to tissue either through a single applicator or through an array of two or three applicators. One of the four 915 MHz output connectors, here output connector 312, is provided as a single connection output connector for use when a single 915 MHz microwave applicator is used. The single 915 MHz applicator is connected to 915 MHz microwave power output connector 312 by a coaxial cable, not shown. Three of the four 915 MHz output connectors, here output connectors 314, 316, and 318, are provided as multiple connection output connectors for connection of 915 MHz applicators to either two or three of the multiple output connectors 314, 316, and 318. The power splitter is an impedance matched splitter circuit which automatically allows the power to be directed to the connection of 1, 2, or 3 applicators while maintaining an impedance match between the 915 MHz signal generator 300 and the one, two, or three applicators connected to the 915 MHz output connectors to provide efficient power transfer from the generator to the applicator or applicators. If more than three applicators are desired for an array, additional power splitters can be included in the system. For example, if two power splitter circuits are used, both power splitter circuits can be connected in parallel to 915 MHz signal generator 300 and anywhere between one and six 915 MHz applicators can be connected to the system.

The signal generators are controlled by a system computer controller 320 which is connected to 2450 MHz generator 302 by interface control and monitoring cable 322 and to the 915 MHz generator 300 by interface control and monitoring cable 324. Signals from the computer controller 320 to each of the generators control operation of the generators such as whether a particular generator is turned on or off, and if turned on, the power level of the output signal produced by the generator and whether the generator is providing output signals to the appropriate output connector or connectors. The generators provide signals back to the computer controller to indicate such things as forward power level, i.e., the power level of the output signals produced by the generator, and reflected power levels, i.e., the level of any power reflected back to the generator. A control interface 326, such as a display screen and keyboard or a touch screen, is connected, such as by cable 328, to the computer controller to supply information from the computer controller to a system operator and to supply information and instructions from a system operator to the computer controller. The computer controller is also connected to the 915 MHz power splitter 308 by interface cable 330 to allow monitoring by the computer controller of the 915 MHz output connectors and of any applicators connected to the 915 MHz output connectors and of any sensor signals supplied through the microwave power cables from the applicators to the power splitter. As described in connection with the applicators described in the parent applications, which applicators may be used as the 915 MHz applicators in the present system, sensor signals from temperature sensors associated with the applicator, and indicative of the operation of the applicator cooling system, may be transmitted over the microwave power cables from the applicators to the power splitter, and which signals are then separated from the power signals and sent to the computer controller. If other 915 MHz applicators are used, various other signals may be provided by such applicators.

Various sensors, such as temperature sensors inserted into the tissue to be heated, may be used with the system. Temperature sensors would indicated the tissue temperature at the position of the sensor and the temperature measured may be used as a feedback control signal for the system, such as for controlling the amount of power applied to the tissue to control the temperature of the heated tissue. If such a temperature sensor is positioned in the tissue at or near the outer margin of the tissue desired to be heated, such temperature sensor can indicate when the normal tissue to be protected is close to being overheated so protective measures need to be taken, such as a reduction of applied power or a system shut down. When sensors are used which provide signals through wires separate from the microwave power cables, such sensor wires can be connected through connectors 332 to a sensor module 334 which can interpret such sensor signals and provide information to the computer controller through interface cable 336, or such signals may be connected directly to the computer controller. While two sensor connectors 332 are shown, any number of sensor connectors may be provided. When one or more of the applicators used in the system are cooled, such as by circulating cooling fluid through such applicators as described in the parent applications, the system may include a Cooling System 340. The various connections between the cooling system and the applicators are not shown in FIG. 1, but may be as described in the parent applications. The system also includes a power supply 342 which supplies power to each of the system components needing power such as the signal generators, the computer controller, the control interface, and if included in the system, the cooling system. The power is supplied to the system components by wire connections between the power supply and the particular components, with such connections not shown in FIG. 1. Further, the power supply will be connected to an outside source of power such as a normal 110 volt utility electrical plug.

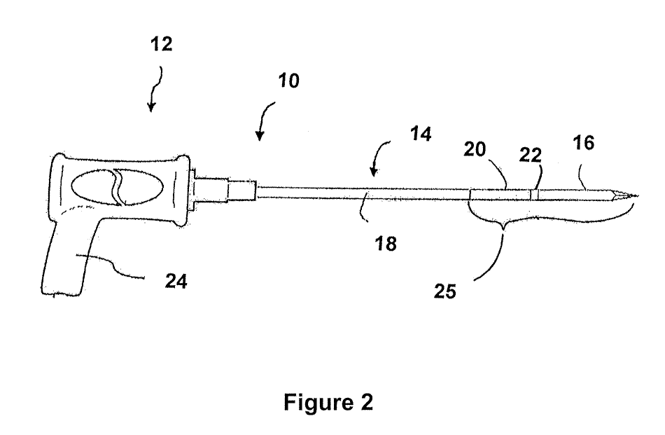

As indicated, any applicator designed for use with 2450 MHz microwave power signals may be used with the microwave energy supply system of the invention to apply the 2450 MHz microwave energy to tissue to be treated and any applicator designed for use with 915 MHz microwave power signals may be used with the microwave energy supply system of the invention to apply the 915 MHz microwave energy to tissue to be treated. Also, as indicated, an applicator suitable for use as the one or more 915 MHz applicator in the system of the invention is described in the parent applications and may include any of the features described in such applications. FIGS. 2, 3, 4, and 5, which correspond to FIGS. 1, 2, 4, and 5 of each of the parent applications, show an embodiment of an applicator that can be used as the one or more 915 MHz applicator in the system of the current invention. Referring to FIGS. 2, 3, and 4, such an applicator, referred to generally as 10, FIG. 2, includes a handle 12 from which a substantially rigid elongate applicator body 14 extends with an insertion tip 16 forming the insertion end portion of the applicator for insertion into a tissue region of the living body. The substantially rigid elongate applicator body 14 includes an outer conductive sleeve 18 extending from the handle 12, a conductive shunt 20, the conductive insertion tip 16, and a dielectric collar 22 positioned between the insertion tip 16 and the shunt 20. As can be seen, dielectric collar 22 joins the conductive insertion tip 16 to both shunt 20 and through shunt 20 to outer conductive sleeve 18. The outside diameters of the exposed portions of the outer conductive sleeve 18, the conductive shunt 20, the dielectric collar 22, and the insertion tip 16 (which may be sharpened at its insertion end 17), are all about equal so as to form a smooth continuous elongate applicator body for insertion into the living body tissue. The elongate applicator body may be coated with a stick resistant dielectric material such as Teflon, not shown. A pistol grip 24 allows the handle to be easily held for manipulation of the applicator.

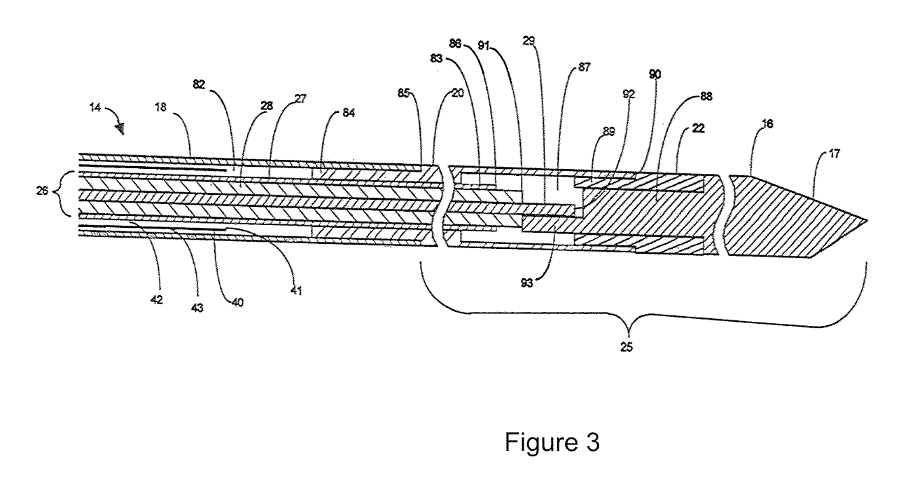

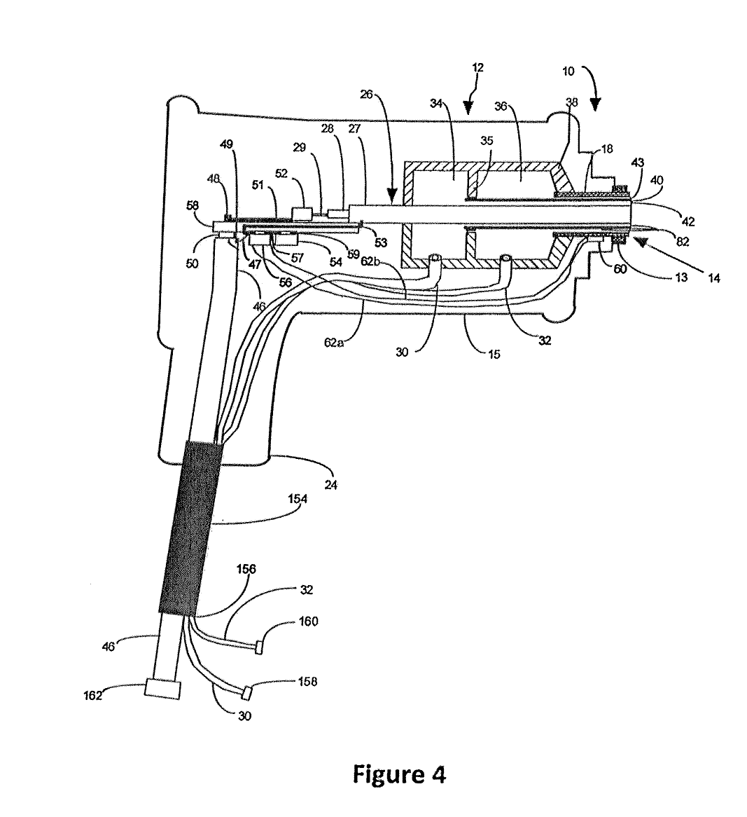

The applicator has a microwave antenna portion 25 toward the insertion tip of the elongate applicator body 14 to radiate microwave energy from the antenna portion into the living body tissue. Microwave energy is transmitted from the handle 12 through the elongate applicator body to the antenna portion by a coaxial microwave transmission line 26, FIGS. 3 and 4, within the elongate applicator body and having an inner conductor 29 and an outer conductor 27 separated by a dielectric material 28 positioned therebetween. Although not required, the coaxial transmission line 26 may be a semirigid coaxial cable with copper inner and outer conductors and a Teflon or Teflon and air dielectric material. No outer dielectric insulating material is used. Such coaxial cable will usually have about a fifty ohm impedance which provides a good impedance match to the microwave generator and to typical living body tissue characteristics.

The outer diameter of the coaxial transmission line (also the outer diameter of the outer conductor 27 of the coaxial transmission line) is smaller than the inside diameter of the outer conductive sleeve 18 so a space 82 is provided between the transmission line and the outer conductive sleeve. This space will be referred to as a cooling fluid space. Conductive shunt 20 is positioned around and in electrical contact with both the insertion end portion 83 of the transmission line outer conductor 27, and the outer conductive sleeve 18. Shunt 20 includes a reduced outer diameter end portion 84 toward the handle end of the applicator dimensioned to fit into the space 82 between the outside surface of the outer conductor 27 of the coaxial transmission line 26 and the inside surface of the outer conductive sleeve 18. With this connection, shunt 20 closes or blocks cooling fluid space 82 toward the insertion end 85 of the outer conductive sleeve 18.

As shown in FIG. 2, elongate applicator body 14 extends from handle 12. As shown in FIG. 4, outer conductive sleeve 18 is secured in the forward portion 13 of handle body 15 and in the forward end of cooling fluid reservoir 38, which cooling fluid reservoir 38 is mounted within handle body 15. Cooling fluid reservoir 38 includes two reservoir chambers 34 and 36 separated by guide sleeve 40 that extends from connection to reservoir partition 35 into outer conductive sleeve 18 and within outer conductive sleeve 18 toward the insertion end of the applicator. The guide sleeve 40 may be a thin walled plastic sleeve made of polyimide plastic such as Kapton. Attachment of the outer conductive sleeve 18 to handle body 15 and fluid reservoir 38, and attachment of guide sleeve 40 to reservoir partition 35, may be with glue, epoxy, or other bonding agent. Coaxial transmission line 26 extends through cooling fluid reservoir 38 and into guide sleeve 40. Coaxial transmission line 26 extends through the entire length of guide sleeve 40 and beyond the guide sleeve insertion end 41, FIG. 3, and into shunt 20.

As seen in FIGS. 3 and 4, guide sleeve 40 extends into cooling fluid space 82 between the outside of coaxial transmission line 26 and the inside of outer conductive sleeve 18. Guide sleeve 40 divides cooling fluid space 82 into an inner cooling fluid space 42 and an outer cooling fluid space 43 along the length of guide sleeve 40 in space 82. Inner cooling fluid space 42 is formed between the outside surface of coaxial transmission line 26 and the inside surface of guide sleeve 40 and outer cooling fluid space 43 is formed between the outside surface of guide sleeve 40 and the inside surface of outer conductive sleeve 18. Reservoir chamber 34 communicates with inner cooling fluid space 42 and reservoir chamber 36 communicates with outer cooling fluid space 43.

While either reservoir chamber 34 or 36 could be a cooling fluid inlet or cooling fluid outlet, it has been found for ease of placement of the temperature sensor, as will be explained in respect of the location of temperature sensor 60, that reservoir chamber 34 can be the cooling fluid inlet reservoir and reservoir chamber 36 can be the cooling fluid outlet reservoir. The inlet and outlet functions however can be reversed with similar performance for tissue cooling. In the arrangement shown, cooling fluid to the applicator will flow from a source of cooling fluid, such as the Cooling System 340 of FIG. 1, through tubing 30 into reservoir chamber 34. From reservoir chamber 34, cooling fluid flows through inner cooling fluid space 42 along the outside surface of coaxial transmission line 26 to cool the outside surface of coaxial transmission line 26. As previously indicated in regard to FIG. 3, cooling fluid space 82 into which guide sleeve 40 extends is blocked at the insertion end portion of outer conductive sleeve 18 by the reduced diameter portion 84 of shunt 20 which fits into and blocks the insertion end of space 82. As seen in FIG. 3, the insertion end 41 of guide sleeve 40 ends before reaching the end of space 82 created by shunt 20 so as to leave an undivided fluid space portion which connects the inner cooling fluid space 42 and the outer cooling fluid space 43. Thus, as cooling fluid flowing in inner cooling fluid space 42 toward the insertion end of the applicator reaches the insertion end 41 of guide sleeve 40, it flows into the undivided space 82 around the insertion end 41 of guide sleeve 40 into outer cooling fluid space 43 and flows along the inside surface of outer conductive sleeve 18 back into reservoir chamber 36 and out fluid outlet tube 32 back to the Cooling System 340 to be cooled and recirculated or flows to a fluid drain.

As shown in FIG. 4, microwave energy is provided to the applicator from a microwave generator or microwave energy supply system, such as the microwave energy supply system shown in FIG. 1, by a coaxial microwave energy supply cable 46 that provides a path for the microwave energy from the microwave energy supply system to the applicator. The coaxial microwave energy supply cable 46 is typically a flexible fifty ohm coaxial cable containing an inner or center conductor 48, an outer conductor 49, and a dielectric spacer 50 therebetween. Coaxial microwave energy supply cable 46 terminates in a cable connector 162, which, depending on the length of cable 46 extending from handle 24, is adapted to connect to a microwave output connector such as one of the 915 MHz output connectors 312, 314, 316 or 318 of FIG. 1, or to connect to a further flexible microwave energy supply cable forming an extension of coaxial microwave energy supply cable 46 which would then connect to a microwave output connector 312, 314, 316 or 318.

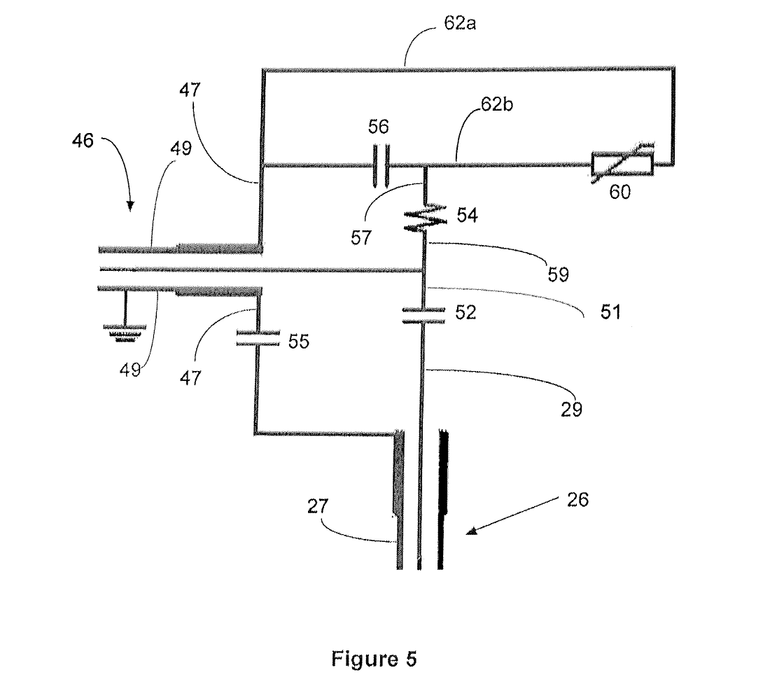

In the illustrated embodiment, the connection between the flexible coaxial microwave energy supply cable 46 and the semi-rigid coaxial transmission line 26 is provided through a coupling circuit on a printed circuit card 58 which supports small chip capacitors and a resistor, (see also FIG. 5 which is a circuit diagram of the circuitry of FIG. 4). The coaxial microwave energy supply cable center conductor 48 is connected by conductive metal path 51 on the circuit card 58 to capacitor 52 which is connected to inner conductor 29 of coaxial transmission line 26. The coaxial microwave energy supply cable outer conductor 49 is connected by conductive element or wire 47 to conductive metal path 53 on the circuit card 58 which is connected to outer conductor 27 of coaxial transmission line 26. This provides a direct path for the microwave currents to flow between the outer conductors. Circuit diagram FIG. 5 shows a capacitor 55 connected between the two outer conductors 49 and 27 which is not necessary and not shown in FIG. 4, but may be advantageous to include to provide further isolation of the microwave antenna from dc currents in the flexible coaxial microwave energy supply cable 46.

A temperature sensor in the form of a thermistor 60 is placed over the outer conductive sleeve 18 and bonded to it so that it is approximately the same temperature as the outer conductive sleeve 18. Thermistor 60, when placed at the location shown in FIG. 4, measures the temperature of outer conductive sleeve 18 at about its handle end, which will be at approximately the temperature of the cooling fluid after flowing through the elongate applicator body 14. Thermistor 60 can be located at other locations that enable it to indicate the approximate temperature of the cooling fluid after or during flow through the applicator, or, if desired, could be located with respect to the applicator to measure tissue temperature of either normal tissue to be protected or of tissue to be heated which is located adjacent to the applicator. When located as shown, thermistor 60 measures the approximate temperature of the cooling fluid between guide sleeve 40 and the outer conductive sleeve 18 as the cooling fluid returns to the cooling fluid outlet reservoir chamber 36 after flowing through inner and outer cooling fluid spaces 42 and 43. The cooling fluid at this location will have reached approximately its highest temperature. Thermistor 60 could be located in the cooling fluid itself, if desired, such as in cooling fluid outlet reservoir chamber 36. The function of this thermistor 60 is to provide an indication that the cooling fluid is actually flowing inside the applicator whenever the microwave power is applied. During the application of microwave energy, the microwave energy causes self heating of the coaxial transmission line 26 in the applicator. This increases the temperature of coaxial transmission line 26 thereby heating the surrounding parts between thermistor 60 and coaxial transmission line 26. Without circulation of cooling fluid, applicator outer conductive sleeve 18 can reach temperatures that can damage normal tissue. The flow of cooling fluid inside the applicator along coaxial transmission line 26 and outer conductive sleeve 18 removes much of that generated heat so that thermistor 60 remain cooler when the cooling fluid is flowing than if there is no fluid flow. If fluid flow stops or is restricted, the fluid will heat to a higher temperature than when properly flowing. When properly flowing, the applicator outer conductive sleeve 18 will remain below tissue damaging temperatures.

A thermistor is a resistive electrical device that varies its resistance depending upon its temperature. The two wires 62a and 62b from thermistor 60 are connected across capacitor 56. Wire 62a connects to capacitor 56 and also connects directly to outer conductor 49 of the flexible coaxial cable 46. Wire 62b attaches to the opposite side of capacitor 56 and also to one side of resistor 54 through conductive metal path 57. The other side of resistor 54 connects to conductive metal path 51 via a wire or conductive metal path 59. Thus, thermistor 60 is connected electrically between inner conductor 48 and outer conductor 49 of flexible coaxial cable 46. This enables the resistance of the thermistor 60 to be monitored by a direct electrical current that is passed from the center conductor 48 through conductive metal traces 51 and 59 to resistor 54 and conductive metal trace 57 and wire 62b to thermistor 60 and back via wire 62a and wire 47 to the outer conductor 49 of flexible coaxial cable 46. Capacitor 52 prevents the direct electrical current from flowing into inner conductor 29 of coaxial transmission line 26 and therefore prevents the direct electrical current from flowing into the applicator antenna and living body into which the applicator is inserted. If capacitor 55 is provided in the circuit, it prevents the direct electrical current from flowing into the outer conductor 27 of coaxial transmission line 26 to further ensure that direct electrical current does not flow into the antenna and into the living body into which the applicator is inserted. This described circuitry allows the flexible coaxial microwave energy supply cable to serve a dual purpose. The dc current for monitoring of the resistance of thermistor 60 passes through the flexible coaxial microwave energy supply cable 46 along with the microwave energy that flows through the flexible coaxial microwave energy supply cable 46 from the microwave generator or microwave energy supply system to the applicator. With the arrangement described, and with the coaxial microwave energy supply cable extending from the applicator connected to a 915 MHz output connector 312, 314, 316, or 318, the dc temperature indicating signal is carried between the thermistor and 915 MHz Power Splitter 308 of the system of FIG. 1 over the same two coaxial cable conductors 48 and 49 that carry the microwave power from the 915 MHz output connector 312, 314, 316, or 318 to the applicator. This eliminates the need for separate additional wires from the handle of the applicator to the system of FIG. 1 to carry the temperature signals from the thermistor.

As indicated, the signal from the thermistor 60 provides an indication to the system controller of the temperature of the outer conductive sleeve and the cooling fluid circulating in the applicator. With the microwave power applied to the applicator, which results in heating of coaxial transmission line 26, as long as cooling fluid is properly flowing in the applicator, the temperature of thermistor 60 will remain low. If the cooling fluid stops flowing in the applicator or flow is restricted for some reason, the coaxial transmission line 26 will begin to heat and the temperature of outer conductive sleeve 18 and of any non-flowing or slowly flowing fluid in the applicator will also increase. This increases the temperature of thermistor 60. This increase in measured temperature of thermistor 60 provides an indication that cooling fluid is not flowing properly, and, in response, the microwave energy supply system can activate an alarm or activate other corrective action.

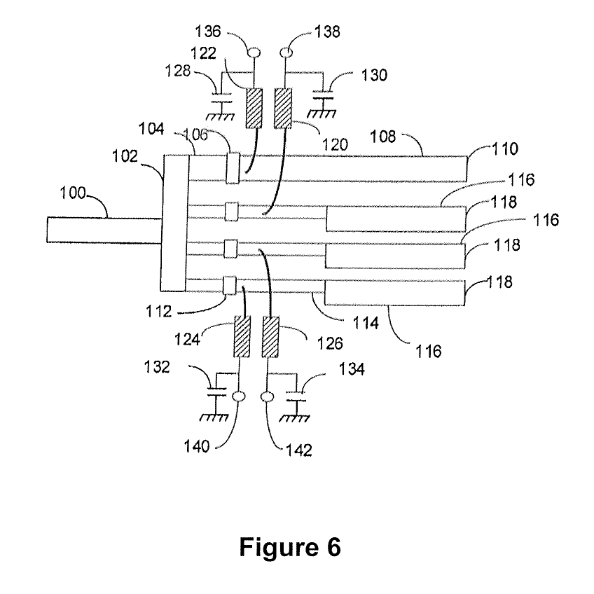

FIG. 6 shows an embodiment of a power splitter circuit according to the invention which is the same as shown and described in FIG. 8 of each of the parent applications. The power splitter embodiment shown in FIG. 6 not only provides impedance matching for the connected applicators, but also adds and separates substantially de sensor signals, such as the described temperature sensor signals, from the microwave power signals and is thus also referred to as a multiplexer. When used as the power splitter 308 in the microwave energy supply system of FIG. 1, the separated temperature or other sensor signals are sent from power splitter 308 to computer controller 320 through interface cable 330. These temperature and/or other sensor signals can be used as feedback signals or alarm signals in operation of the microwave energy supply system.

With the illustrated embodiment of the microwave energy supply system of FIG. 1, microwave power signals from 915 MHz signal generator 300 are supplied to power splitter circuit 308 through coaxial cable 310, shown as coaxial cable 100 in FIG. 6. This cable 310 (100 in FIG. 6) will generally be of fifty ohm impedance. The multiplexer and power splitter circuit as shown in FIG. 6 is generally provided on a printed circuit card made of low loss dielectric material such as Teflon based material with a ground plane on one side and the circuit show in FIG. 6, that represents the conductive paths forming various transmission lines, on the other side. The input microwave power signal from cable 310 (cable 100 in FIG. 6) connects to an input in the form of a conductive patch 102 that provides a power splitting section. This simultaneously directs microwave power to four paths, one single path shown by transmission line 104, capacitor 106 in transmission line 104, and output portion transmission line 108, and three identical paths shown by transmission lines 114, capacitors 112 in transmission lines 114, and output portion transmission lines 116. In the single path, capacitor 106 in transmission line 104 is a chip type capacitor that conducts microwave power but blocks substantially direct current signals to prevent substantially direct current signals from reaching power splitting patch 102. The input microwave power flows from conductive patch 102, along transmission line 104 with capacitor 106, and through output transmission line portion 108 to circuit output port 110, which corresponds to 915 MHz microwave power output connector 312 in FIG. 1. The transmission lines 104 and 108 are fifty ohm transmission lines which together have an electrical length delay of one hundred eighty degrees at the 915 MHz microwave operating frequency. Capacitor 106 has a low impedance of typically less than two ohms reactive impedance to avoid mismatching the transmission line. This then directs microwave power from the input transmission line 100 to the circuit output port 110, i.e., to 915 MHz microwave power output connector 312. Output port 110 (output connector 312) forms an output port for connection of a single 915 MHz applicator through a flexible fifty ohm impedance coaxial microwave energy supply cable attached to 915 MHz microwave power output connector 312. This output port 110, which is sometimes referred to herein as a single connection output port or single connection output connector, is used if only a single 915 MHz applicator is to be connected to the multiplexer and power splitter circuit. This provides impedance matching between the 915 MHz microwave signal generator 300 and a single 915 MHz microwave applicator.

Power splitter conductive patch 102 is also connected to the three identical paths, each formed by a transmission line 114 with a series chip capacitor 112 therein and transmission line output portions 116. Similarly to capacitor 106, each capacitor 112 is a chip type capacitor having a low impedance of typically less than two ohms reactive impedance to conduct microwave power but to block substantially direct current signals to prevent substantially direct current signals from reaching power splitting patch 102. The input microwave power flows from conductive patch 102, along transmission lines 114 with capacitors 112, and through output transmission lines 116 to circuit output ports 118, which correspond to 915 MHz microwave output connectors 314, 316, and 318 in FIG. 1. These output ports 118, which are sometimes referred to herein as a multiple connection output ports or multiple connection output connectors, are used if multiple 915 MHz applicators are to be connected to the multiplexer and power splitter circuit. The electrical length delay of a transmission line 114 from the power splitter conductive patch 102 through the capacitor 112 to output transmission line 116 is approximately ninety degrees at the 915 MHz microwave operating frequency. Also the characteristic impedance of a microwave transmission line 114 with capacitor 112 is typically between seventy and ninety ohms from the power splitter conductive patch 102 to the end of transmission line 114, which is typically a quarter of a wavelength long (a ninety degree delay length), to provide impedance matching for the input when either two or three applicators are connected to the multiple connection output ports 118. The electrical length delay of an output transmission line 116 is approximately ninety degrees at the 915 MHz microwave operating frequency. Therefore, the transmission lines 114 and 116 together have an electrical length delay of approximately one hundred eighty degrees at the 915 MHz microwave operating frequency. Also the characteristic impedance of an output transmission line 116 is about fifty ohms to provide impedance matching for a fifty ohm impedance flexible coaxial microwave energy supply cable connected to an output port 118 and to a 915 MHz applicator. Each of these three identical paths direct microwave power from the input transmission line 100 to one of the circuit output ports 118, i.e., to one of the 915 MHz microwave power output connectors 314, 316, or 318. Output ports 118 (output connectors 314, 316, and 318) form output ports for connection of multiple, here either two or three, 915 MHz applicators, each through a flexible fifty ohm impedance coaxial microwave energy supply cable attached to either two or three of the 915 MHz microwave power output connector 314, 316, and 318. These output ports 118 are used only when multiple 915 MHz applicators are to be connected to the multiplexer and power splitter circuit. These paths provide impedance matching between the 915 MHz microwave signal generator 300 and multiple 915 MHz microwave applicators.

The described power splitter circuit of FIG. 6 forms an impedance matched microwave power splitter that when a single applicator is to be used, the single applicator is connected to the single connection output to port 110 (915 MHz output connector 312). When this is the case the other three output ports (915 MHz output connectors 314, 316, and 318), each a multiple connection output port 118, are not connected to an applicator. The path length from the power splitter conductive patch 102 to each of these multiple connection output ports 118 is one hundred eighty degrees. The microwave power that travels to each of these multiple connection output ports 118 is reflected completely back when there is no connection of an applicator to the ports and this reflected power is reflected with the same phase angle as the incoming power to these ports because this is an open circuit termination. This means that the overall phase delay of the power from the power splitter conductive patch 102 to each of the multiple connection output ports 118 and back to the power splitter conductive patch 102 is three-hundred-sixty degrees. This unique phase delay then appears to the power splitter as an open circuit. Thus, the open ports 118 turn these paths into tuning paths that do not reflect power that would reach the input line 100, but would direct the full power only to single connection output port 110 (output connector 312 of FIG. 1) and to the single applicator that is connected to output port 110 for efficient power transfer to the single applicator.

When two or three applicators are connected to respective multiple connection output ports 118, there will be no applicator connected to the output port 110. The path delay between the power splitter conductive patch 102 and the output port 110 is also one-hundred-eighty degrees. Therefore, the delay to the output port 110 and back to the conductive patch 102 is three-hundred-sixty degrees. Therefore, when there is no applicator attached to the single connection output port 110 it becomes a tuning path for the microwave energy. (Also, if only two applicators are connected to multiple connection output ports 118, the path to the output port to which an applicator is not connected also acts as a tuning path for the microwave energy). The result is that the microwave power splitter circuit of FIG. 6 is an impedance matched splitter which automatically allows the power to be directed to the connection of 1, 2, or 3 applicators. It would not be permitted to attach only a single applicator to one of the multiple connection output ports 118 because, although all paths without an applicator attached would still become tuning paths, the impedance of the transmission lines 114 are chosen to provide an impedance match for the microwave generator when using either two or three applicators, so such connection of an applicator to only a single multiple connection output port would result in an impedance mismatch and would cause unacceptable reflected power to the input line 100. Also, as apparent from the above description, if no applicators are connected to any of the ports (output connectors) of the power splitter circuit, all transmission paths appear as open circuits. This allows multiple power splitter circuits to be use to provide for more than three applicators when desired. For example, if two power splitter circuits are connected in parallel, anywhere between one and six applicators can be connected to the system.

When the power splitter circuit of FIG. 6 is also used to separate substantially direct current sensor signals from microwave signals, the power splitter circuit also includes an inductive coil or choke 120, 122, 124, and 128 connected to each of the transmission lines 104 and 114. Each of these inductive coils is connected through a capacitance to the ground chassis with capacitors 128, 130, 132, and 134, respectively. These capacitors and the inductive coils filter the microwave signals from temperature sensing ports 136, 138, 140, and 142, but pass substantially direct current signals from the transmission lines 104 and 114 to these temperature sensing ports. These temperature sensing ports can be connected to temperature monitoring circuitry in a temperature module or, as shown in FIG. 1, are connected directly to the system computer controller 320 which provides temperature monitoring functions. Thus, the computer controller 320 will receive the separated dc sensor signals indicating the measured resistance of the thermistors, or indicating other variables that may be sensed and represented by such sensor signals, and use such signals as feedback signals in control of the system and/or to indicate alarm conditions. For example, if, rather than a temperature sensor, the sensor is a tissue electrical resistance sensor, it would indicate when the heating is sufficient to significantly increase the tissue resistance indicating that the tissue at the location of the sensor has been coagulated to the point that the fluids have been boiled or driven away with the heating. This feedback would indicate that the tissue ablation and coagulation at that position has been accomplished and lead to the possible termination of the applied power to prevent charring or additional charring of the tissue at that location or would provide an indication of how much additional time or power may be expected to be necessary to complete the treatment of the remaining target tissue. If the signals are direct current temperature sensor signals from the applicators, they provide a measurement to the system computer controller of the temperature measured by the temperature sensors in each of the applicators. If the applicators used are those of the parent applications as described above, such signals enable the measurement of applicator cooling temperature to determine that fluid is properly flowing in each of the connected applicators to cool the applicators and protect the normal body tissues.

When a temperature sensor or other resistance is provided in the 915 MHz applicators so as to provide direct current temperature sensing signals from the applicators to the temperature sensing ports, such signals can also provide an indication to the system computer controller of whether applicators are connected to particular output ports of the power splitter circuit. If an applicator is connected to a particular power splitter circuit output port, for example to output port 110, a temperature signal will be present on temperature sensing port 136. The system computer controller will then know that an applicator is connected to output port 110. Similarly, if a temperature signal is present on temperature sensing ports 138 and 142, the system computer controller will know that two applicators are connected to two of the multiple connection output ports 118 and will be able to identify which of the two output ports have applicators connected thereto. If the system computer controller senses temperature signals on temperature sensing ports 136 and 138, the system computer controller knows that there are two applicators connected to the power splitter circuit, but that the applicators are not properly connected since one of the two applicators is improperly connected to single connection output port 110 while the other of the two applicators is properly connected to one of the multiple connection output ports 118. The system controller can then provide a warning signal to a system user indicating that the applicators are improperly connected, and that the applicator connected to the single connection output port 110 should be disconnected and connected to one of the multiple connection output ports 118. In a similar way, if the sensor is a tissue resistance sensor, the sensor resistance signals will indicate that the sensor and an applicator to which the sensor is attached, is connected to a particular system connector.

If temperature or other sensing is not required, but the sensing of the attachment of microwave applicators to power splitter circuits is desired, the thermistor, other temperature sensors, or other sensors, such as a tissue resistance sensor, that provide direct current temperature signals, can be replaced with regular resistors which will provide substantially dc signals in the manner of thermistor to indicate that microwave applicators are attached to a power splitter output port and indicate to which port or ports the applicators are attached. This use of resistors will be considered equivalents of the thermistors or other sensors that provide direct current sensor signals for the purposes of the applicator detection.

The example embodiment of the multi frequency microwave energy supply and coagulation system described above describe the use of two frequencies and describe the two frequencies as two microwave frequency signals of 2450 MHz and 915 MHz. These frequencies were chosen as examples because these two frequencies are generally approved frequencies for medical treatment purposes in most countries of the world and because these two approved frequencies can provide significantly different heating patterns during tissue heating with presently available applicators. It should be realized, however, that where different frequencies are approved for medical use and where there are advantages to being able to select between two or more frequencies for treatment either during single treatment procedures or during different treatment procedures, such other frequencies may be used either in place of one or the other or both of the described 2450 MHz and 915 MHz or in addition to one or the other or both of the described 2450 MHz and 915 MHz. Further, the multi frequency microwave energy supply system of the invention can be used in connection with any electromagnetic energy therapy, not just ablation or coagulation therapy, with any electromagnetic energy signal applicators appropriate for the desired therapy. When a 2450 MHz single microwave channel is provided, it may take the place of the single channel connection 915 MHz connector previously described and all 915 MHz connectors may be multiple connection 915 MHz connectors for use with 915 MHz phased array applicators to provide both the small spherical ablation of 2450 MHz and the larger spherical ablations of the 915 MHz synchronous phased array.

Further, the generator that has been described as a 2450 MHz microwave signal generator for generating the electromagnetic energy signals for the single applicator can be replaced by a radiofrequency generator that has a frequency typically lower than 40 MHz where the heating currents from an inserted electrode, which can be similar in construction with the microwave antenna or with other standard radiofrequency applicator electrodes, can be used as the single applicator electromagnetic energy signal source. The antenna configuration can serve as radiofrequency bipolar electrodes. It is also common for radiofrequency electrode energy sources to have a return current grounding electrode. Such a multifrequency generator system can provide significant clinical advantages for the clinician to provide several methods from which to optimize the heating distribution to match the target tissue to be treated with the same treatment system.

In addition, one or more of the described electromagnetic energy signal generators can be a variable frequency energy generator rather than a substantially single frequency generator. However, broad frequency band energy generators typically have poor efficiency. Therefore, use of individual high efficiency electromagnetic energy signal generators with different operational frequencies is the preferred configuration.

Whereas the invention is here illustrated and described with reference to embodiments thereof presently contemplated as the best mode of carrying out the invention in actual practice, it is to be understood that various changes may be made in adapting the invention to different embodiments without departing from the broader inventive concepts disclosed herein and comprehended by the claims that follow.

1. A system for providing electromagnetic energy signals of at least one selected frequency of a plurality of frequencies available for selection to at least one applicator for insertion into living body tissue for heat treatment of diseased tissue within the living body tissue, the system comprising:
a first generator for generating electromagnetic energy signals of a first frequency;
a second generator for generating electromagnetic energy signals of a second frequency;
a first connector associated with the first generator and adapted for connecting a first applicator to receive electromagnetic energy signals from the first generator;
at least one second connector associated with the second generator and adapted for connecting at least one second applicator to receive electromagnetic energy signals from the second generator; and
a system controller for coordinating operation of the first and second generators to provide electromagnetic energy signals, when desired, from the first generator to the first connector and from the second generator to the at least one second connector to provide a desired heat treatment to the diseased tissue within the living body tissue.

2. A system for providing electromagnetic energy signals of at least one selected frequency of a plurality of frequencies available for selection to at least one applicator according to claim 1, wherein the at least one second connector is a plurality of second connectors for connecting one or a plurality of second applicators to receive electromagnetic energy signals from the second electromagnetic energy signal generator.

3. A system for providing electromagnetic energy signals of at least one selected frequency of a plurality of frequencies available for selection to at least one applicator according to claim 2, wherein the first frequency is 2450 MHz and the second frequency is 915 MHz.

4. A system for providing electromagnetic energy signals of at least one selected frequency of a plurality of frequencies available for selection to at least one applicator according to claim 2, wherein the system controller coordinates operation of the first and second generators to provide both electromagnetic energy signals from the first generator and from the second generator during a single treatment procedure to provide the desired heat treatment to the diseased tissue.

5. A system for providing electromagnetic energy signals of at least one selected frequency of a plurality of frequencies available for selection to at least one applicator according to claim 4, wherein the system controller coordinates operation of the first and second generators to provide electromagnetic energy signals to the first and second connectors at different times to avoid simultaneous application of electromagnetic energy signals at both the first and second frequencies.

6. A system for providing electromagnetic energy signals of at least one selected frequency of a plurality of frequencies available for selection to at least one applicator according to claim 5, wherein the system controller coordinates operation of the first and second generators to alternately provide electromagnetic energy signals to the first and second connectors at a rate to provide heating of tissue which simulates simultaneous heating by signals of both the first and second frequencies.

7. A system for providing electromagnetic energy signals of at least one selected frequency of a plurality of frequencies available for selection to at least one applicator according to claim 6, wherein the system controller coordinates operation of the first and second generators to switch between providing signals to the first and second connectors during respective first and second preset time periods.

8. A system for providing electromagnetic energy signals of at least one selected frequency of a plurality of frequencies available for selection to at least one applicator according to claim 7, wherein the respective first and second preset time periods are each less than about 20 seconds.

9. A system for providing electromagnetic energy signals of at least one selected frequency of a plurality of frequencies available for selection to at least one applicator according to claim 4, wherein the system controller coordinates operation of the first and second microwave generators to provide microwave signals to the first and second connectors simultaneously when desired to apply microwave signals to tissue at both the first and second frequencies simultaneously.

10. A system for providing electromagnetic energy signals of at least one selected frequency of a plurality of frequencies available for selection to at least one applicator according to claim 2, wherein the plurality of second connectors includes a single connection second connector for use when a single second applicator is to be connected to the at least one second connector, and two or more multiple connection second connectors for use when two or more second applicators are to be connected to the two or more multiple connection second connectors, and wherein the single connection second connector is impedance matched and tuned to provide efficient energy transfer when a single second applicator is to be used and the multiple connection second connectors are impedance matched and tuned to provide efficient energy transfer when a number of second applicators anywhere from two to the total number of multiple connection second connectors provided are to be used.

11. A system for providing electromagnetic energy signals of at least one selected frequency of a plurality of frequencies available for selection to at least one applicator according to claim 10, wherein the single connection second connector and the multiple connection second connectors are provided by a power splitter circuit between the second generator and the at least one second connector.

12. A system for providing electromagnetic energy signals of at least one selected frequency of a plurality of frequencies available for selection to at least one applicator according to claim 11, wherein the power splitter circuit includes one single connection second connector and three multiple connection second connectors to allow attachment of a single second applicator to the one single connection second connector, two second applicators to two of the multiple connection second connectors, or three applicators to three of the multiple connection second connectors.

13. A system for providing electromagnetic energy signals of at least one selected frequency of a plurality of frequencies available for selection to at least one applicator according to claim 12, wherein the impedance through the power splitter circuit for the single connection second connector is about fifty ohms with an electrical length delay of about one hundred eighty degrees, and the impedance through the power splitter circuit for each of the three multiple connection second connectors is configured in two sections, an input section having an impedance of between about seventy and ninety ohms with an electrical length delay of about ninety degrees, and an output section having an impedance of about fifty ohms with an electrical length delay of about ninety degrees.

14. A system for providing electromagnetic energy signals of at least one selected frequency of a plurality of frequencies available for selection to at least one applicator according to claim 11, wherein the system includes a plurality of power splitter circuits connected to the second generator to allow more than three second applicators to be connected to the system.

15. A system for providing electromagnetic energy signals of at least one selected frequency of a plurality of frequencies available for selection to at least one applicator according to claim 11, wherein the power splitter circuit is also a multiplexer circuit for separating substantially DC signals from the electromagnetic energy signals.

16. A system for providing electromagnetic energy signals of at least one selected frequency of a plurality of frequencies available for selection to at least one applicator according to claim 15, wherein the multiplexer circuit for separating substantially DC signals from the electromagnetic energy signals separately separates substantially DC signals from electromagnetic energy signals for each of the second connectors.

17. A system for providing electromagnetic energy signals of at least one selected frequency of a plurality of frequencies available for selection to at least one applicator according to claim 16, wherein the separated substantially DC signals are transmitted to the system controller.

18. A system for providing electromagnetic energy signals of at least one selected frequency of a plurality of frequencies available for selection to at least one applicator according to claim 17, wherein the system controller is programmed to determine to which second connector an applicator is connected, receipt of a substantially DC signal from a second connector indicating an applicator is connected to that second connector, and wherein the controller is programmed to determine from a determination of the number of applicators connected to the multiplexer and power splitting circuit and the particular second connectors to which the applicators are connected, whether when only a single second applicator is detected, the single applicator is properly connected to the single connection second connector and whether when a plurality of second applicators are detected, the plurality of second applicators are properly connected to only the multiple connection second connectors, and to provide an output signal indicating if the applicators are not properly connected.

19. A system for providing electromagnetic energy signals of at least one selected frequency of a plurality of frequencies available for selection to at least one applicator according to claim 18, additionally including at least one second applicator having means for generating a substantially DC signal and providing the substantially DC signal to the at least one second connector.

20. A system for providing electromagnetic energy signals of at least one selected frequency of a plurality of frequencies available for selection to at least one applicator according to claim 19, wherein the means for generating a substantially DC signal is a temperature sensor associated with the applicator.
